(19) United States
(12) Patent Application Publication
(10) Pub. No.: US 2001/0026920 A1
(43) Pub. Date: Oct. 4, 2001

(54) Internal standards and controls for multiplexed assay

(76) Inventors: Mark Chandler, Austin, TX (US); Michael Spain, Austin, TX (US)

Correspondence Address: PEPPER HAMILTON LLP, 600 Fourteenth Street, N.W., Washington, DC, 20005-2004, US

(21) Appl. No.: 09/791,894

(22) Filed: Feb. 26, 2001

Related U.S. Application Data
(60) Provisional application No. 60/270,161, filed on Feb. 22, 2001.
(60) Provisional application No. 60/185,132, filed on Feb. 25, 2000.

(51) Int. Cl. C12Q 1/68; G01N 33/53; G01N 33/537; G01N 33/543

(52) U.S. Cl. 435/6; 435/7.92; 436/518

(57) ABSTRACT

Internal standards and controls are provided for detecting and/or compensating for sources of measurement error in multiplexed diagnostic and genetic assays. The internal standards include subsets of particles comprising ligand binding partner specific for analytes of interest, each subset having a different known amount of ligand binding partner. Internal controls include subsets of particles comprising ligand binding partner chosen to provide information relating to high-dose hook effects, dilutional linearity, interfering assay factors, or sample or reagent omission.

### 1. REFERENCES

This application claims the benefit of priority from Provisional Application entitled “Internal Standards for Multiplexed Assay,” filed Feb. 22, 2001 by Mark R. Chandler and Michael D. Spain, which is herein incorporated by reference, and also claims benefit of priority from Provisional Application Ser. No. 60/185,132, filed Feb. 25, 2000.

### 2. FIELD OF THE INVENTION

The present invention relates generally to methods of, and products for, compensating for or detecting sources of sample anomalies. The present invention relates, more specifically, to internal assay calibration of multiplexed diagnostic and genetic analysis of clinical specimens.

### 3. BACKGROUND OF THE INVENTION

Analysis of clinical specimens is important in science and medicine. Multiplexed assays to determine qualitative and/or quantitative characteristics of a specimen are known in the art. For example, U.S. Pat. No. 5,981,180 (the “'180 patent”), which is hereby incorporated by reference, discloses methods, instrumentation, and products for detecting multiple analytes in a fluid sample by flow cytometric analysis. The products include bead subsets, each bead subset having a different reactant bound to the bead. The individual subsets are prepared so that beads within a subset are relatively homogenous but differ in at least one distinguishing characteristic from beads in any other subset. Therefore, the subset to which a bead belongs can readily be determined after beads from different subsets are pooled. The methods include pooling the variously labeled subsets prior to assay and mixing the pooled beadset with a fluid sample to test for analytes reactive with the various reactants bound to the beads.

Diagnostic and genetic analysis can be subject to measurement errors. Assay-to-assay variations in the standardization of analytic systems can cause major increases in down stream medical costs. Causes of shifts in distributions of the clinical test values are changes in assay calibration and lot-to-lot differences in reagent manufacturing. For example, factors leading to measurement error include: instrument calibration error, samples that include substances which interfere with the assay, reagent (including sample) omission, and the hook effect.

### 4. SUMMARY OF THE INVENTION

The present invention relates to methods of, and products for, internally calibrating multiplexed assays, which can provide a more robust assay system having minimal assay variation. The term “calibrating” is understood to mean compensating for measurement error and/or detecting sources of measurement error. The term “multiplexed assay” is understood to mean an assay that can detect and/or measure multiple targets. The term “target” is generally understood to mean any substance desired to be analyzed, including analytes, internal standards, internal controls, or any component of the assay reagant mixture. The term “analyte” is understood to mean any substance suspected of being present in a sample. The term “internal,” when used in conjunction with standard or control, e.g. “internal standard” or “internal control,” is understood to mean capable of being included in an assay reagent mixture or in any sample. Thus, similarly, the phrase “internally calibrating” is understood to mean the use of internal standards and controls for calibrating assays.

In one aspect, the present invention provides internal standards which can be used to at least partially compensate for measurement errors in multiplexed assays. In some embodiments, the internal standards are substances comprising a detectable, calibrated amount of analyte. In some embodiments, the internal standards comprise subsets of substances, wherein each subset comprises a different, detectable, calibrated amount of analyte. In other embodiments, an internal standard may be a subset of particles adapted for use in methods according to the '180 patent. The particles may comprise a microsphere or bead coupled to an amount of target analyte. The particles in each subset have one or more characteristic classification parameters that distinguish the particles of one subset from those of another subset. The parameter may at least be the amount of target analyte bound to the microsphere or bead.

In another aspect, the present invention provides methods of at least partially compensating for measurement errors in multiplexed assays. In some embodiments, the method comprises adding at least one subset of internal standards to a reagent mixture or sample suspected of including the target analyte or analytes, analyzing the at least one subset of internal standards to generate at least one calibration point, and measuring the amount of target analyte, or analytes, present in the sample by reference to the at least one calibration point. In some embodiments, the method comprises adding at least two subsets of internal standards to a reagent mixture or sample suspected of including the target analyte or analytes, analyzing the at least two subsets of internal standards to generate an internal standard curve, and measuring the amount of analyte, or analytes, present in the sample by reference to the generated internal standard curve.

In another aspect, the present invention provides internal controls which can be used to detect sources of measurement error in multiplexed assays. In some embodiments, the internal controls can be used to detect omission of a sample or reagant. Internal controls for detecting omissions can comprise at least one ligand capable of coupling with a component expected to be present in the sample or reagent mixture, wherein the internal controls are capable of being detected when the target component is coupled with the ligand. In some embodiments, the internal controls can be used to detect the presence of interfering substances. Internal controls for detecting interfering substances can comprise at least one ligand capable of coupling with specific interfering substance suspected of being present in the sample or reagent mixture, wherein the internal controls are capable of being detected when target interfering substance is coupled with the ligand. In some embodiments, the internal controls can be used to detect high-dose hook effects. Internal controls for detecting high-dose hook effect can comprise a low concentration of at least one ligand capable of binding analyte, wherein the internal controls generate a detectable signal proportional to the amount of bound analyte. In some embodiments, the internal controls can be used to verify linearity of response. Internal controls for verifying linearity can comprise at least one subset of internal controls, wherein each subset can comprise a ligand capable of binding analyte, wherein the subsets are distinguishable at least by the amount of analyte which can be bound, and wherein the internal controls are detectable when analyte is bound to the ligand. The various internal controls can be a subset of particles adapted for use in methods according to the '180 patent. The particles in each subset have one or more characteristic classification parameters that distinguish particles of one subset from those of another subset. Accordingly, for example, internal controls for detecting omission can be distinguished from internal controls for detecting high-dose hook effect and thus both internal controls can be used simultaneously in a multiplexed assay.

In another aspect, the present invention provides methods of detecting sources of measurement error in multiplexed assays. In some embodiments, the method comprises adding internal controls for detecting omission of a sample or reagent to the reagent mixture. In some embodiments, the method comprises adding internal controls for detecting interfering substances to the reagant mixture. In some embodiments, the method comprises adding internal controls for alerting a user to possible high-dose hook effect to the reagent mixture. In some embodiments, the method comprises adding internal controls for testing linearity of response to the reagent mixture.

It will be apparent to one of ordinary skill in the art that specific embodiments of the present invention may be directed to one, some or all of the above-indicated aspects as well as other aspects, and may encompass one, some or all of the above- and below-indicated embodiments as well as other embodiments. Thus, for example, a method according to the present invention may comprise adding internal standards to compensate for measurement error, whereas another method according to the invention may comprise both adding internal standards to compensate for measurement error and adding internal controls to detect the presence of interfering substances.

### 5. BRIEF DESCRIPTION OF THE DRAWINGS

FIG. 1 is a conceptual representation of one embodiment according to the present invention.

FIG. 2 is a conceptual representation of another embodiment according to the present invention.

FIG. 3 is a conceptual representation of another embodiment according to the present invention.

FIG. 4 is a conceptual representation of another embodiment according to the present invention.

FIG. 5 is a conceptual representation of another embodiment according to the present invention.

FIG. 6 is a conceptual representation of another embodiment according to the present invention.

FIG. 7 is a conceptual representation of other embodiments according to the present invention.

### 6. DETAILED DESCRIPTION OF SPECIFIC EMBODIMENTS

The present invention relates generally to methods of and products for internally calibrating multiplexed assays using internal standards and/or internal controls. In some embodiments of the invention the internal standards and/or internal controls are subsets of particles. The term “particle” refers a microsphere or bead coupled to at least one ligand for use in flow cytometric multiplexed assays, for example in accordance with U.S. Pat. No. 5,981,180. The term “subset of particles” refers to a group of particles sharing essentially the same characteristic classification parameters. By “essentially” it is meant that the particles are similar to the extent that they can be identified as belonging to the same group of particles and also distinguished from the particles of another group. The term “ligand ” refers to any substance capable of coupling with at least one other substance.

A general method using internal standard and/or control particles to internally calibrating a multiplexed assay can comprise:

(a) exposing a mixture comprising a pooled population of at least one subset of particles to a reagent mixture comprising at least one type of signal ligand, wherein the particles within a subset are: (i) similarly-sized; (ii) exhibit at least one characteristic classification parameter that distinguish the particles of one subset from those of another; and (iii) comprise a tracer or a ligand binding partner chosen to couple with a desired target ligand, the ligand binding partner associated with one subset of particles may be the same as or different from the ligand binding partner associated with another subset of particles, and the target ligand is chosen from analytes suspected of being in a sample, tracers, and the at least one signal ligand, provided either that: (i) a specific target ligand is an analyte chosen from interfering factors and characteristic sample components excluding the analyte or analytes of interest, or (ii) the particles of at least one subset comprise a known concentration of a specific ligand binding partner corresponding to an analyte of interest, the concentration is chosen to test for a hook effect, or (iii) in one or more groups of subsets of particles, the particles in the subsets of the group comprise the same tracer or a ligand binding partner corresponding to the same target ligand but at known concentrations that vary with each member of the one or more selected groups;

(b) passing the exposed particles through an examination zone;

(c) collecting data as the exposed particles pass through the examination zone relating to: (i) one or more characteristic classification parameters of each particle including data on signal, for example fluorescence emission, intensities, and at least either (ii) the presence or absence of a complex formed between the ligand binding partner and target ligand or (iii) the apparent concentrations of the one or more particular analytes of interest associated with each member of the one or more selected groups, or both (ii) and (iii); and

(d) substantially simultaneously with collecting, classifying each particle according to its subset and, when the target ligand is an analyte, quantifying an amount of analyte associated with each subset.

The term “analyte of interest” refers to the analyte or analytes desired to be analyzed. The term “signal ligand ” refers to a ligand which is capable of being detected. A signal ligand can be, for example, any substance having associated therewith a detectable label such as a fluorescently- or radioactively-tagged antibody or antigen. The term “ligand binding partner” refers to a ligand capable of coupling with a target ligand. According to the general method described above, the ligand binding partner is typically conjugated to a particle. However, a “ligand binding partner” can more generally be a ligand capable of coupling with a target ligand. The term “target ligand ” refers to a ligand in (or put into) the reagent mixture or sample, which is capable of coupling with a ligand binding partner. For example, an antibody associated with a particle and capable of coupling with an antigen in a sample is a “ligand binding partner,” while the antigen is a “target ligand.” As another example, an antigen associated with a particle and capable of coupling with a signal ligand is a “ligand binding partner,” while the signal ligand is a “target ligand.” The term “tracer” refers to a signal ligand that competes with a target ligand for binding to a particular ligand binding partner. For example, if a fluorescently-labeled antigen “A” and its unlabeled antigen “A” counterpart both compete for binding to a particular antibody associated with a particle, the flourescently-labeled antigen is a “tracer,” the unlabeled antigen is a “target ligand,” and the antibody is a “ligand binding partner.” The term “interfering factor” relates to any substance in the sample or reagent mixture which can interfere with the coupling of a ligand binding partner and a target ligand leading to an artificially low result.

Some embodiments according to the above-described general method are directed to methods of compensating for certain measurement errors that can occur in performing sandwich immunoassays. In those embodiments, a first set of particles comprising beads or microspheres coupled to a first ligand binding partner are used to measure the concentration of a target ligand, which target ligand is, in this case, the analyte of interest in the patient specimen. The first ligand binding partner is capable of coupling with the analyte of interest. One or more additional sets of particles, comprising microspheres or beads coupled to various concentrations of the same analyte, serve as internal standards. The same signal ligand capable of coupling with the analyte and the same signal measurement systems are used to measure all sets of particles. The concentrations of the analyte coupled to the different internal standard particle sets are chosen to give signals approximately equal to the signals of the standard curve associated with particle set one. Factors for converting the signals measured on the internal standard particle sets to the signal measured with first set of particles are determined by measuring human specimens having known analyte concentrations established using reference methods.

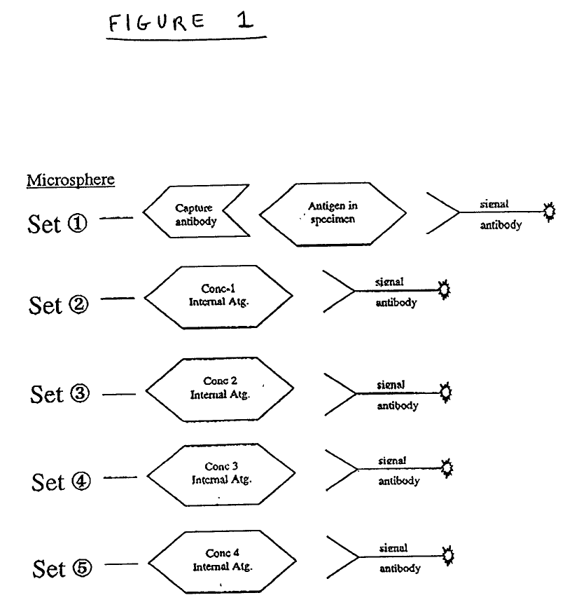

FIG. 1 is a conceptual representation of an example of a method relating to sandwich immunoassay embodiments. Subset 1 in FIG. 1 represents a set of first particles. A first particle comprises an amount of first ligand binding partner coupled to a microsphere or bead. In this embodiment of the invention, the ligand binding partner is a capture antibody capable of coupling with the target ligand analyte. Set 2 in FIG. 1 represents a set of second particles. A second particle comprises a first amount of ligand standard coupled to a microsphere or bead. Set 3 in FIG. 1 represents a set of third particles. A third particle comprises a second amount of ligand standard coupled to a microsphere or bead. Set 4 in FIG. 1 represents a set of fourth particles. A fourth particle comprises a third amount of ligand standard coupled to a microsphere or bead. Set 5 in FIG. 1 represents a set of fifth particles. A fifth particle comprises a fourth amount of ligand standard coupled to a microsphere or bead. The ligand standard in particle sets two through five is the target ligand analyte. According to the embodiment represented in FIG. 1, the target ligand analyte is an antigen. It is understood that the term “antigen” refers to any substance capable of forming a complex with an antibody. The signal ligand in this embodiment is a signal antibody.

The amounts of antigen coupled to particle sets two through five are chosen to give signals, when coupled to the signal ligand, approximately equal to the standard curve for signals generated by particle set one when forming a sandwich complex with target ligand analyte and signal ligand. The standard curve for signals generated by particle set one generally reflects measurements corresponding to typical concentrations of target analyte in specimens of interest, for example, in samples of human body fluid. Thus, for example, an amount of antigen coupled to the second set of particles is chosen so that the second particle-signal antibody complexes produce signal approximately equal to a measurement along the standard curve and an amount of antigen coupled to the third set of particles is chosen so that the third particle-signal antibody complexes produce signal approximately equal to another measurement along the standard curve.

The subsets of particles are mixed into an aqueous specimen sample suspected of containing target antigen, along with the aqueous signal antibody under conditions suitable for promoting formation of sandwich complexes of capture antibody-antigen-signal antibody and complexes of internal standard antigen-signal antibody. Flow cytometric multiplexed assay methods, which preferably only analyze signal generated by the sandwich complexes of capture antibody-antigen-signal antibody and complexes of internal standard antigen-signal antibody, are then used to distinguish the complexes and read signals generated by the complexes. A internal standard curve can be derived from signal generated by the sets of internal standard antigen-signal antibody complexes. The amount of antigen can then be determined by comparing the signal generated by the set of capture antibody-antigen-signal antibody sandwich complexes to the derived internal standard curve.

Some embodiments according to the above-described general are directed to methods of compensating for certain measurement errors that can occur in performing competitive immunoassays. According to this aspect of the present invention, limited amounts of a first ligand binding partner are immobilized on particle set one. Signal ligand (in this case also known as “tracer”) and the target ligand analyte, an analyte endogenous to the specimen, compete for binding to limited sites on the first ligand binding partner. After removal of unbound tracer, the signal is quantitated and compared to a standard curve to quantitate the target ligand analyte concentration in the specimen. One or more additional sets of particles, comprising various concentrations of the same tracer immobilized on microspheres or beads, are used to generate an internal standard curve. The concentrations of the immobilized tracer are chosen to mimic the standard curve associated with particle set one. The standard curve reflects measurements corresponding to typical values associated with concentration of target analyte in specimens of interest, for example, in samples of human body fluid. Again, factors for converting the signals measured with the additional internal standard particle sets to the signal measured with particle set one are determined by measuring specimens, for example specimens from humans, having known analyte concentrations established using reference methods.

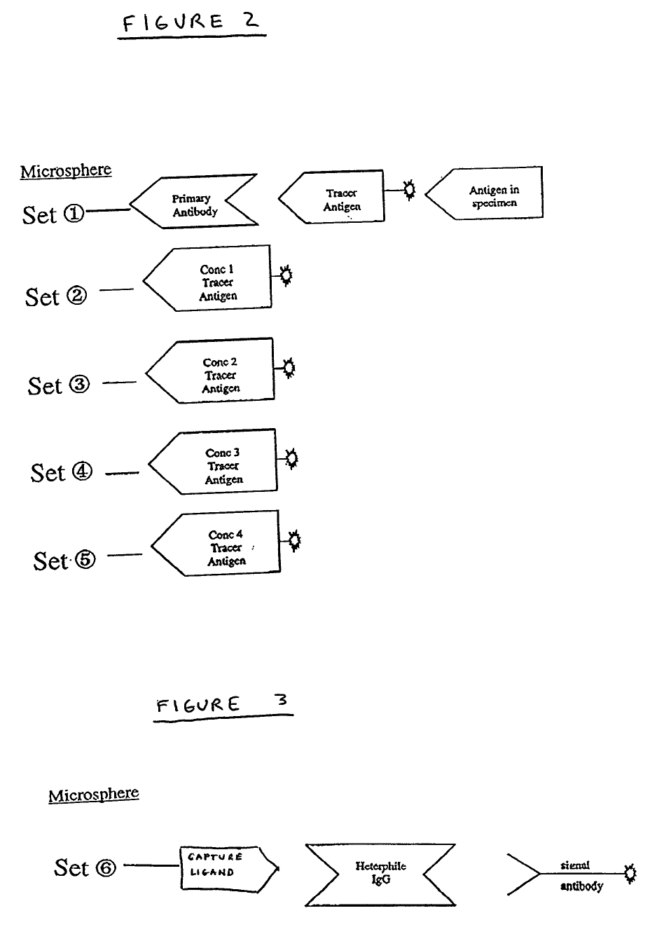

FIG. 2 is a conceptual representation of an example of a method relating to competitive immunoassay embodiments. Subset 1 in FIG. 2 represents a set of first particles. A first particle comprises an amount of first ligand binding partner immobilized on a microsphere or bead. In this embodiment of the invention, the first ligand binding partner is a primary antibody capable of coupling with either a tracer or a target analyte in a competitive manner. In this example, the tracer is a fluorescently-tagged antigen and the target analyte is also an antigen. Tracer antigen and target antigen compete for binding to the limited sites on the antibody. The tracer antigen and target analyte antigen may be the same antigen. Subset 2 in FIG. 2 represents a set of second particles. A second particle comprises a first amount of the same tracer immobilized on a microsphere or bead. Subset 3 in FIG. 2 represents a set of third particles. A third particle comprises a second amount of the tracer immobilized on a microsphere or bead. Subset 4 represents a set of fourth particles. A fourth particle comprises a third amount of the tracer immobilized on a microsphere or bead. Subset 5 in FIG. 2 represents a set of fifth particles. A fifth particle comprises a fourth amount of the tracer immobilized on a microsphere or bead. According to this embodiment, the tracer in sets two through five is an antigen having associated therewith a detectable label.

The amounts of tracer antigen immobilized on particle sets two through five are chosen to give signals approximately equal to a measurement along the standard curve associated with signals generated by particle set one, when coupled to tracer. The standard curve associated with particle set one generally reflects measurements corresponding to typical values associated with concentration of target antigen in specimens of interest, for example, in samples of human body fluid.

The subsets of particles are mixed into an aqueous specimen sample suspected of containing the target antigen, along with aqueous tracer antigen under conditions suitable to allow formation of complexes of primary antibody-tracer and primary antibody-target antigen. Unbound tracer is removed, and flow cytometric multiplexed assay methods, which preferably only analyze signal generated by the primary antibody-tracer complex and by particle sets two through five, are then used to distinguish the various particle sets and read signals generated by the various particle sets. An internal standard curve can be derived from signal generated by the particle sets two through five. The signal measurement generated by particle set one can then be evaluated by comparing that signal to the internal standard curve.

Some embodiments according to the above-described general method are directed to methods for compensating for certain measurement errors that occur in performing gene probe assays. The methodology for gene probe assays is similar to the technique for sandwich immunoassays exemplified in FIG. 1 and discussed more generally above, with synthetic oligonucleotides being substituted for the internal antigens and nucleotide primers being substituted for the capture and signal antibodies. Oligonucleotides are synthesized with an irrelevant sequence for attachment at the bead end and with a sequence capable of being hybridized to an oligonucleotide detection probe located elsewhere within the oligonucleotide. These sequences can be used to control for the efficiency of hybridization for a specific sample. To control for batch to batch variation, capture probes specific for irrelevant nucleic acid sequences can be attached to the beads. For example, if one is trying to detect nucleic acids from human sources, the use of capture probes for plant specific (irrelevant) nucleic acids would be attached to specific subsets of beads to generate the standardization signals. Batch to batch testing results can be normalized based upon the levels of signals obtained from the irrelevant sequences.

Other embodiments of the above-described general method are directed to methods for detecting errors that can occur as a result of the presence of interfering factors in the assay system. In immunoassays, interfering factors can affect coupling between the analyte and ligand binding partners. As a result the detected signal corresponds to an amount of analyte that is lower than the actual amount of analyte in the sample. According to this aspect of the present invention, internal controls can be used to identify whether interfering factors are present in the assay system.

FIG. 3 is a conceptual representation of an example of an assay relating to detection of interfering factors. Subset 6 in FIG. 3 represents a set of sixth particles. A sixth particle comprises a microsphere or bead coupled to a ligand binding partner capable of binding an interfering factor, the target ligand. In the exemplified embodiment, the interfering factor is human anti-heterophile antibodies. A sixth particle set can be included, along with signal ligand capable of binding human anti-heterophile antibodies, in a reagent mixture for a multiplexed assay. Signal can be detected when sandwich complexes of ligand binding partner-interfering factor-signal ligand form. Accordingly, if signal generated by particle six is detected in the multiplexed assay, it is an indication that interfering factors, in the particular illustrated embodiment human anti-heterophile antibodies, are present in the sample.

Other embodiments according to the above-described general method are directed to methods for identifying measurement errors that can occur as a result of reagent omission. For example, if an operator inadvertently neglects to add sample to the reagent mixture, a test will inaccurately indicate a lack of target ligand analyte in the sample. According to this aspect of the present invention internal controls can be used to verify that various components have been added to the reagent mixture.

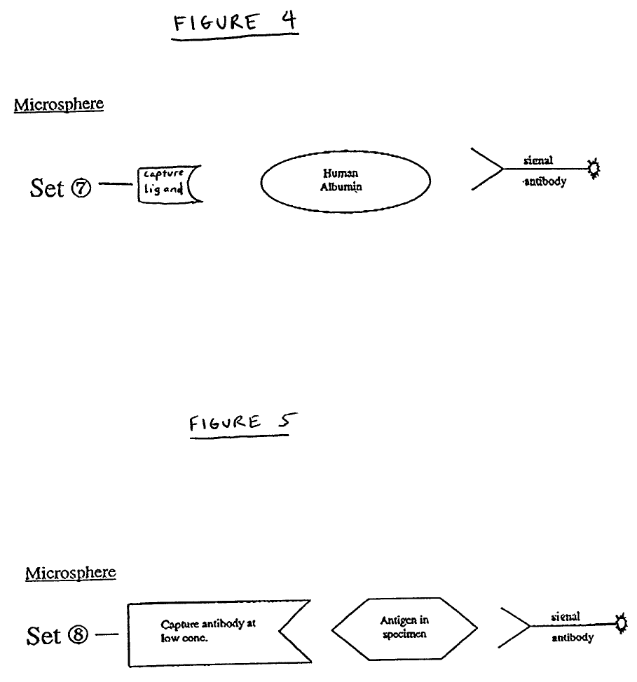

FIG. 4 is a conceptual representation of an example of a method relating to reagent omission embodiments. Subset 7 in FIG. 4 represents a sets of seventh particles. A seventh particle comprises a microsphere or bead coupled to a ligand binding partner capable of binding human albumin, the target ligand. Because human albumin is present in most human blood samples, particle set seven can be used as a control to verify that the blood sample is included in the reagent mixture in assays for analytes in blood.

According to this embodiment of the present invention, particle set seven can be included, along with a signal ligand capable of binding human albumin, in a multiplexed assay used to detect analytes in human blood. Signal is detected when sandwich complexes of ligand binding partner-human albumin-signal ligand form. Accordingly, if signal generated by particle set seven is detected in the multiplexed assay, it is an indication that sample has been added to the reagent mixture.

Other embodiments of the above-described general method are directed to methods for identifying measurement errors due to the hook effect. According to those embodiments, internal control standards can be used to identify whether the assay is being performed in the hook region.

The hook effect can be understood in relation to the standard sandwich assay. The hook effect becomes significant in such assays when very large target ligand concentrations are present. In such situations, there is so much target ligand present in the sample that all available combining sites on the first ligand binding partner as well as those available on the signal ligand are filled with the available target ligand. Indeed, there may still be additional unattached target ligands available. As a result, of the plethora of target ligands available fewer sandwich complexes are being formed since only some of the ligand binding partners and signal ligands will be attached to the same target ligand. Consequently, an increasing target ligand concentration results in a proportional increase in immobilized signal ligand until the target ligand concentration becomes so great that fewer sandwich complexes are formed whereupon the curve rapidly drops off giving a false, lower concentration of target ligand.

FIG. 5 is a conceptual representation of an example of an assay relating to detection of hook effect. Subset 8 in FIG. 5 represents a set of eighth particles. An eighth particle comprises a microsphere or bead with a known amount of ligand binding partner. In the illustrated embodiment, the ligand binding partner is the capture antibody of FIG. 1 and therefore subset eight can be used along with subsets two through five of FIG. 1 in the same sandwich assay. The known amount is chosen to test for hook effect. For example, the amount can be a low concentration of ligand binding partner. Subset eight coupled with lower concentrations of the capture antibody normally would give low signals; however, in the presence of very high concentration of target antigen, this subset would give a higher signal. Such a result can be used to alert the user of potential high-dose hook effects.

As another example, the eighth particle can comprise merely a known amount of capture antibody. If the signal measured from the subset of eighth particles is lower than expected, that is lower than the signal typically associated with the chosen known amount, such a result could also alert the user to potential high-dose hook effects.

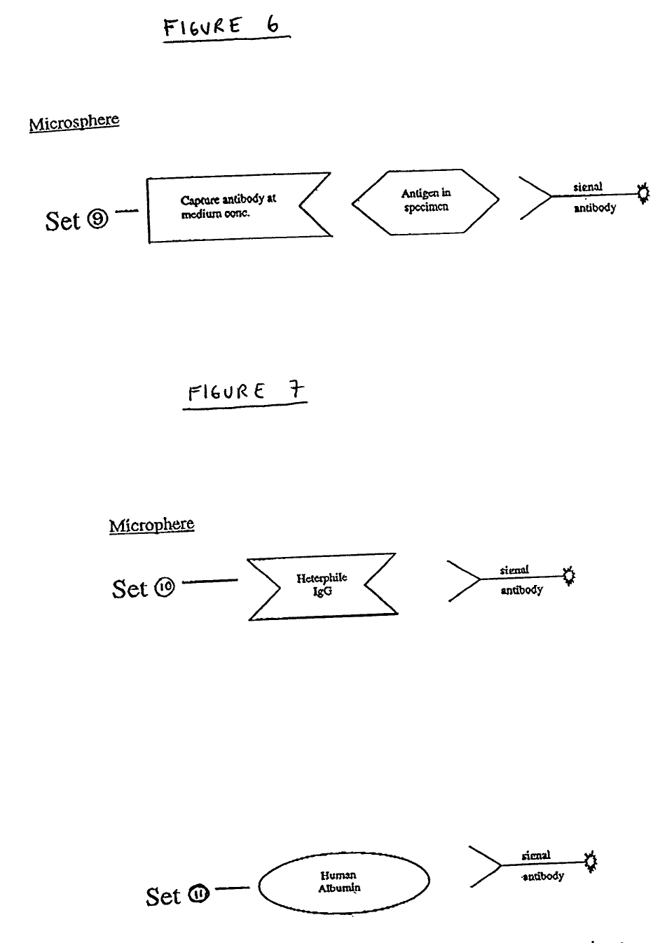

Other embodiments according to the above-described general embodiment are directed to methods for verifying linearity of response. FIG. 6 is a conceptual representation of an example of a method directed to verifying linearity. In the embodiment illustrated in FIG. 6, additional sets of particles (only one is illustrated but more may be used) are included in the sandwich assay of FIG. 1. The additional sets of particles comprise microspheres or beads with different concentrations of the same capture antibody used in subset 1 of FIG. 1. By using those additional sets of particles, the system can verify that the antigen in the specimen reads in the same manner on the reference antigen (dilutional linearity).

Another aspect of the present invention are internal standards products useful for the methods according to the present invention. Embodiments of such products have been described above and include, for example, subsets of particles, wherein each subset comprises microspheres or beads coupled to different concentrations of the target analyte.

Another aspect of the present invention are internal control products for use with methods according to the present invention. Embodiments of such products have been described above and include, for example, a subset of particles comprised of microspheres or beads coupled to a ligand capable of coupling to human albumin.

Another aspect of the present invention is kits for the detection or quantitation of an analyte or analytes. The kits can comprise one or more sets of ligand binding partner, each set is distinguishable from other sets. In some embodiments, the kits comprise one or more sets of particles, each set being distinguishable from other sets, for example by its fluorescent signature. The particles are coupled to ligand binding partner. The particles, apart from the ligand binding partner, can be polymeric particles which range in size from 0.01 to 1000 micrometers (μm) in diameter. In one embodiment, the size ranges from 0.1-500 μm. In another embodiment the size ranges from 1-200 μm. In another embodiment the size ranges from 2-12 μm. The particles can be similarly-sized. By “similarly-sized,” it is meant that difference between particles within a set is not more than 15%. The particles can be made of any regularly shaped material. In one embodiment, the shape is spherical. However, particles of any other shape can be employed. The shape of the particle can serve as an additional distinction parameter, which can be discriminated by flow cytometry, e.g., by high-resolution slit-scanning.

The kits can include sets of particles for use as internal standards. Or else the kits can includes a set or sets of particles for use as controls. Or else the kits can include sets of particles for use as internal standards and a set or sets of particles for use as controls. The kits can also include signal ligands for use with sandwich or competitive immunoassays. The kit may also contain a binding partner for the signal ligand which forms a complex with for example, an antibody, antigen, biotin, hapten, or analyte.

A person of ordinary skill will appreciate that changes could be made to the embodiments described above without departing from the broad inventive concept thereof. It is understood, therefore, that this invention is not limited to the particular embodiments disclosed, but it is intended to cover modifications within the sprit and scope of the invention.

For example, the present invention also encompasses internal controls for background signal, and methods of using those internal controls. As represented conceptually in FIG. 7, several sets of microspheres can also be used to control for assay interferences, such as human anti-heterophile and non-specific binding to microspheres without specific antibody attached (i.e. using human albumin as negative control).

As another example, when the present invention is applied to sandwich immunoassays, as discussed above in connection with FIG. 1, the ligand binding partner and signal ligand may be antigens rather than antibodies, and the target ligand can therefore be an antibody rather than an antigen. More generally, the ligand binding partner and signal ligand can be any ligand capable of binding target analyte, and in the case of a sandwich immunoassay, the ligand binding partner and signal ligand should be capable of simultaneously binding target analyte. Non-limiting examples of potential target analytes include peptides, polypeptides, proteins (such as enzymes, glycoproteins, lipoproteins and avidin and including antibodies and antigenic proteins), hormones (such as thyroxine, triiodothyronine, human chorionic gonadotropin, estrogen, ACTH and substance P), immune system modulators (such as interleukin-1, interleukin-6 and tumor necrosis factor), tumor markers (prostate specific antigen, CEA, hCG, CA-549 (a breast cancer antigen), and AFP), vitamins, steroids, carbohydrates (such as polysaccharides), glycolipids, lipids, drugs (such as digoxin, phenytoin, phenobarbital, morphine, carbamazepine and theophylline), antibiotics (such as gentimicin), components of cells and viruses (such as Streptococcal species, herpes viruses, Gonococcal species, Chlamydial species, retroviruses, influenza viruses, Prevotella species, Porphyromonas species, Actinobacillus species and Mycobacterium species), nucleic acids (such as single- and double-stranded oligonucleotides), pharmaceuticals, haptens, lectins, biotin, and other materials readily apparent to one skilled in the art. Accordingly, ligand binding partner (or “capture” ligands) and signal ligands will be chosen by their ability to bind to the specific analyte of interest. Thus, for example, the signal ligand might be an antibody or antigen, but it might also be biotin, avidin, hapten, or analyte.

Likewise, the detectable label associate with the signal can be, not only, a fluorescent signal, such as a fluorescent dye, but also, for example, an enzyme, biotin, avidin, isotope, luminescent dye, colloidal gold, a colloidal metal sol, colored dye, phosphorescent dye, or radioisotope such as I¹²⁵ and the like.

Thus too, although analysis of human body fluids are primarily described, the present invention can be adapted for use in analyzing samples of any material which contains a target analyte. For example, the present invention can be adapted for in analyzing samples of air, water, soil, or biological samples of animal, microbial, or plant origin. Biological samples include bodily fluid samples such as urine, serum, plasma, spinal fluid, sputum, whole blood, saliva, urogenital secretions, fecal extracts, pericardial washes, gastric washes, peritoneal washes, pleural washes, clonical washes, nasal/pharyngeal washes, respiratory discharges, and vaginal secretions. The constituents of the biological samples can include lipids, proteins, bilirubin, hemoglobin, immunoglobin, hormones, drugs, antigens, allergens, toxins, tumor markers, soluble cell molecules, nucleic acid, and the like.

Accordingly, a method for measuring immobilized reactants for environmental testing applications aimed at identifying and measuring pesticides and their aminated metabolites are within the scope of the invention. For example, the environmental target analyte could be aldrin, alachlor, atrazine, BAY SIR 8514, S-bioallethrin, chlorosulfuron, cyanazine, 2,4-D, DDT, dichlorfop-methyl, dieldrin, diflubenzuron, endosulfon, iprodione, kepone, maleic hydrazide, metalaxyl, oxfendazole, parathion, paraoxon, paraquat, pentachlorophenol, 2,4,5-T, terbutryn, triadimefon, warfarin. The analyte could also be environmental pollutants such as polychlorinated biphenyls (PCBs), polybrominated biphenyls (PBBs), polynuclear aromatic hydrocarbons (PARs), nitroaromatics, cyclic ketones, BTEX (benzene, toluene, ethyl benzene, and xylene), nitrosamines, haloalkanes, dioxins, dibenzofurans, or TNT.

Another sandwich immunoassay variation could be the use of ligand standards (i.e. the additional subsets of particles useful for generating an internal standard curve) which are not identical to the target ligand. In this embodiment, a signal ligand capable of binding the ligand standards should also be used in the assay. Preferably, the signal generated by the ligand standards particle subsets will still correspond to signal associated with the standard curve for the subset of particles used to measure concentration of target ligand.

As another example, the subsets of particles comprising ligand standard may optionally be calibrated prior to use. Calibration may be accomplished by using commercially available standards to generate a standard curve by reacting the standards with signal ligands capable of binding to the standards and measuring the signal generated by the standard-signal ligand complex. Ligand standard to be used in the assay can first be reacted with signal ligand. The signal generated by the ligand standard-signal binding partner complex can then be measured and compared to the curve derived from measuring the commercially available standards.

Also, although the illustrated embodiments are particularly adapted for use with addressable microsphere technology developed by Luminex Corporation, and disclosed for example in U.S. Pat. No. 5,981,180, the present invention can be adapted for use with any multiplexed assay system.

The disclosed embodiments can also be modified to detect sources of anomalies in addition to high-dose hook effect, sample omission, and heterophile antibodies. For example, the matrix effect and rheumatoid factor are other sources of anomalies. Matrix or serum effects are sample-specific properties that interfere with the measurement of the test result. The matrix effect can be caused by an excess bodily fluid constituents such as lipemia, bilirubinemia, hemoglobinemia, hemolysis, lipids, proteins, hemoglobin, immunoglobin, hormones, drugs, antigens, allergens, toxins, tumor markers, soluble cell molecules, and nucleic acid. These constituents may either increase or decrease the measurement signal, causing an inaccurate result. The internal standard(s) according to an embodiment of the invention can be used in the same aliquot as the test itself. In the case of antigen detection, one microsphere set carries the specific test. For example, in a capture-sandwich immunoassay for thyroid stimulating hormone, one microsphere set would carry capture antibody specific for TSH while a second microsphere set would carry a known amount of TSH on its surface. Patient sample and a specific, labeled reporter antibody (TSH) would be added to the two discrete microsphere sets. Classification and measurement would then be performed. Matrix effects that shift the value of the specific assay would have an equal effect on the “internal standard” microsphere set. Since the internal standard would have a known amount of reactivity in the absence of a matrix effect, any effect present would be detected and the system could “correct” the actual test result for the presence of the effect. In one embodiment, a 5-point standard curve could be included with every test by employing five distinct microsphere sets, each of which had a different amount of analyte on its surface.

Likewise, embodiments of the invention, for examples those directed to detecting sources of error relating to the interfering factor heterophile antibodies described above in connection with FIG. 3, can be modified to detect sources of error corresponding to the presence of rheumatoid factor, and interfering factor. The auto-antibodies or anti-immunoglobulins to immunoglobulin G (IgG) are also known as rheumatoid factors (RF) because of their association with rheumatoid arthritis. RF is also found with varying frequency in patients with most of the connective tissue diseases, many chronic and sub-acute infections, and a variety of miscellaneous disorders. In addition, RF is found in many apparently healthy persons, particularly the elderly. If the capture antibody for an assay is a mouse monoclonal IgG and the patient's has human anti-mouse antibodies (HAMA) or Rheumatoid Factor then either of these can bind to the capture antibody, thus blocking the site and giving an artificially low result. In addition to RF frequent in autoimmune diseases, e.g., polyarthritis, juvenile chronic polyarthritis, ankylozing spondylitis, Reiter's syndrome, there are antinuclear antibodies (ANA), anti-DNA antibodies, antihistone antibodies, acetylcholine receptor antibodies, antierythrocyte antibodies, antiplatelet antibodies, or thyroglobulin antibodies. These compounds can also bind, cross-link, and essentially inactivate reporter antibodies. To detect their presence using the internal controls according to an embodiment of the present invention, one can construct a microsphere set that has nonspecific mouse IgG on its surface or a oligopeptide with affinity to RF. Included in the reporter mixture are labeled human IgG (HAMA is IgG) and IgM (RF is IgM) antibodies. The presence of HAMA or RF should result in detectable label on this nonspecific microsphere set. Human rabbit antibodies could be detected using a different microsphere set or by including mouse and rabbit IgG on the same set.

What is claimed is:

1. A method of internally calibrating a multiplexed assay comprising:
(a) exposing a mixture comprising a pooled population of at least one subset of particles to a reagent mixture comprising at least one type of signal ligand, wherein the particles within a subset are: (i) similarly-sized; (ii) exhibit at least one characteristic classification parameter that distinguish the particles of one subset from those of another; and (iii) comprise a tracer or a ligand binding partner specific to a target ligand, the ligand binding partner associated with one subset of particles may be the same as or different from the ligand binding partner associated with another subset of particles, and the target ligand is chosen from analytes suspected of being in a sample, tracers, and the at least one type of signal ligand, provided either that: (i) a specific target ligand is an analyte chosen from interfering factors and characteristic sample components excluding the analyte or analytes of interest, or (ii) the particles of at least one subset comprise a known concentration of a specific ligand binding partner corresponding to an analyte of interest, the concentration being chosen to test for a hook effect, or (iii) in one or more groups of subsets of particles, the particles in the subsets of the group comprise the same tracer or a ligand binding partner corresponding to same target ligand but at known concentrations that vary with each member of the one or more selected groups;
(b) passing the exposed particles through an examination zone; and
(c) collecting data as the exposed particles pass through the examination zone relating to: (i) one or more characteristic classification parameters of each particle including data on signal intensities, and at least either (ii) the presence or absence of a complex formed between the ligand binding partner and target ligand or (iii) the apparent concentrations of the one or more particular analytes of interest associated with each member of the one or more selected groups, or both (ii) and (iii).

2. A method according to claim 1, further comprising substantially simultaneously with collecting, classifying each particle according to its subset and, when the target ligand is an analyte, quantifying an amount of analyte associated with each subset.

3. A method according to claim 1, wherein the at least one subset of particles is at least two subsets of particles, the particles of a first subset comprise ligand binding partner specific for target ligand chosen from analytes and the particles of at least a second subset comprise ligand binding partner specific for target ligand chosen from signal ligand.

4. A method according to claim 3, wherein the at least one subset of particles is at least five subsets of particles, the particles of the second, third, fourth, and fifth subset of particles comprise ligand binding partner specific for the same signal ligand but at known concentrations that vary with each subset.

5. A method according to claim 1, wherein the at least one subset of particles is at least two subsets of particles, the particles of a first subset comprise ligand binding partner capable of binding a first or a second target ligand, wherein the first target ligand is a tracer and the second target ligand is an analyte, and the particles of at least a second subset comprise a tracer equivalent to the first target ligand.

6. A method according to claim 5, wherein the at least one subset of particles is at least five subsets of particles, the particles of the second, third, fourth, and fifth subset of particles comprise the same tracer but at known concentrations that vary with each subset.

7. A method according to claim 1, wherein the identity of a ligand binding partner is selected to provide information relating to the inclusion or omission of a sample or reagent in the reagent mixture.

8. A method according to claim 1, wherein the signal ligands and tracers comprise at least one fluorochrome and the signal intensities are fluorescence emission intensities.

9. A method according to claim 1, wherein the particles in each subset exhibit two or more characteristic fluorescence emission classification parameters.

10. A method according to claim 9, wherein particles of one subset differ from particles of another subset in an intensity of at least one fluorescence emission classification parameter.

11. A method according to claim 1, wherein one or more analytes is chosen from antigens, antibodies, peptides, proteins, nucleic acid sequences, and enzymes.

12. A method according to claim 2, wherein results of the method are displayed in real time.

13. A method according to claim 12, wherein the results take into account the known and apparent concentrations of the one or more particular analytes associated with each member of the one or more selected groups of subsets of particles.

14. A method according to claim 1, wherein the ligand binding partner is chosen from antibodies, antigens, synthetic oligonucleotides, and capture probes.

15. A method according to claim 1, wherein the identity or amount of ligand binding partner is selected to provide information relating to high-dose hook effects, interfering assay factors, sample or reagent omission, or dilutional linearity.
